(19) United States
(12) Patent Application Publication
(10) Pub. No.: US 2023/0149684 A1
(43) Pub. Date: May 18, 2023

(54) SELECTIVE AND REVERSIBLE BLOOD-BRAIN BARRIER BREAKDOWN BY ULTRASOUND EMISSION

(71) Applicant: BRAINWAVES, Paris (FR)

(72) Inventors: Jean-Claude SADIK, Paris (FR); Aline CRITON, Boulogne-Billancourt (FR)

(73) Assignee: BRAINWAVES, Paris (FR)

(21) Appl. No.: 17/916,659

(22) PCT Filed: Apr. 1, 2021
(86) PCT No.: PCT/FR2021/050575

§ 371 Date: Oct. 3, 2022

(30) Foreign Application Priority Data
Apr. 1, 2020 (FR) 2003270

(51) Int. Cl. A61M 37/00

(52) U.S. Cl. CPC A61M 37/0092; A61N 2007/0039

(57) ABSTRACT

An ultrasound emitting device, configured to selectively and reversibly break the blood-brain barrier of a patient, including a structure configured to be placed on at least one portion of the head of the patient, at least two ultrasound transducers coupled to the structure and configured to emit ultrasounds of diagnostic intensity. Also, a method for improving the bioavailability in brain tissue of at least one inflammation mediator in a patient. Further, a method for treating diseases of the central nervous system using the method for improving the bioavailability of inflammation mediators in a patient in need thereof, and method for treating diseases of the central nervous system using the device. Finally, the use of the device for the treatment of diseases of the central nervous system.

### FIELD OF THE INVENTION

The present invention relates to a device configured to selectively and reversibly break the blood-brain barrier of a subject by emitting ultrasound.

The present invention also relates to a method for improving the bioavailability in brain tissues of at least one agent against inflammation mediators in a subject by selectively and reversibly breaking the blood-brain barrier of a subject.

The present invention also relates to the use of the device of the present invention for the treatment of diseases of the central nervous system.

### PRIOR ART

Mediators of inflammation and particularly Tumor Necrosis Factor (TNF) is an essential component of the brain immune system and plays an important role in regulating nerve pulse transmission. In excess, it can adversely affect the good conduction of the nervous system and cause an inflammatory reaction.

In the central nervous system (CNS), tumor necrosis factor alpha (TNF-alpha) derives from activated microglia and plays an essential role of inflammatory mediator. In particular, it has been shown by brain imaging that intracerebral microglia activation results in intracerebral neuroinflammation after stroke or other forms of brain injury, due to a high level of relaxed TNF-alpha. Several analyses also showed that patients with Alzheimer's disease had a high level of TNF-alpha in their cerebrospinal fluid.

Generally used for the management of chronic disabling inflammatory diseases, such as rheumatoid arthritis, ankylosing spondylitis, severe psoriasis and its rheumatic form, anti-TNF-alpha represents a class of therapeutic agents useful in the treatment of diseases of the central nervous system.

However, in the treatment of diseases of the central nervous system, the blood-brain barrier (BBB) represents a major obstacle. Indeed, the blood-brain barrier is formed by layers of cells lining the cerebral vascular system and thus allowing stability of the brain environment by preventing the entry of many substances, typically of a molecular weight greater than 180 Da, such as toxins, viruses, bacteria and therapeutic agents circulating in the blood.

Thus, it is often observed in the treatment of diseases of the central nervous system that only a very small amount of active principle necessary to treat the pathology reaches through the blood-brain barrier. Known anti-TNF alpha are fusion proteins or monoclonal antibodies, their molecular weight generally exceeds 100 KDa and therefore have difficulty in passing the blood-brain barrier.

Therefore, there is a need to improve the bioavailability of anti-TNF-alpha for use in the treatment of diseases of the central nervous system.

For this purpose, techniques for selectively and reversibly opening the blood-brain barrier have been developed to allow the necessary amount of therapeutic agent to reach brain tissue in a safe and controlled manner.

For example, U.S. Pat. No. 7,896,821 relates to a method and apparatus for reversibly breaking the blood-brain barrier using low intensity focused ultrasound. The application WO 2011/057028 also relates to a method and a device for modulating brain activity in humans using ultrasound. It has not been demonstrated that the device of the application WO 2011/057028 makes it possible to act on the vascular structures of the brain and thus to reversibly break the blood-brain barrier.

However, the prior art transducers used in ultrasonic therapy generally use long wave trains, close to the continuous, which generates strong waves heating within the transducers. It is therefore generally necessary to use a water circulation cooling system, in order to avoid the risks of deterioration of the transducer.

Also, none of the prior art devices have been used for the administration of anti-TNF-alpha for the treatment of diseases of the central nervous system.

The present invention aims to meet this need by proposing a device configured to selectively and reversibly break the blood-brain barrier of a subject by emitting ultrasound.

The device of the present invention can, for example, use micromachined ultrasonic transducers, having low internal mechanical losses compared to the transducers of the prior art and thus a lower temperature increase.

### SUMMARY OF THE INVENTION

The invention relates therefore to a device configured to selectively and reversibly break the blood-brain barrier of a subject by emitting ultrasound, comprising: a structure configured to be placed on at least a portion of the head of a user; and at least two ultrasonic transducers coupled to said structure and configured to emit ultrasound with diagnostic intensity. In one embodiment, said device is adapted to increase the bioavailability in brain tissues of at least one agent against inflammation mediators. In a preferred embodiment, the agent for controlling inflammation mediators is at least one anti-inflammatory agent. In a still more preferred embodiment, the agent for controlling inflammation mediators is at least one anti-TNF alpha.

In one embodiment, the at least two ultrasonic transducers are of capacitive micromachined, piezoelectric micromachined or piezoelectric type.

In one embodiment, the said structure consists of a helmet, a cup, a hood or a headband.

In one embodiment, the at least two ultrasonic transducers are positioned in a frontal plane and/or a sagittal plane.

In one embodiment, the at least two ultrasonic transducers are movable along a frontal axis and/or a sagittal axis.

In one embodiment, the device further comprises a control device coupled to the at least two ultrasonic transducers and configured to control the frequency and power of the ultrasound emitted by the at least two ultrasonic transducers.

In one embodiment, the at least two ultrasonic transducers rest on the surface of the scalp.

In one embodiment, the device comprises a plurality of ultrasonic transducers forming an array of transducers on the surface of the scalp.

In one embodiment, the at least two ultrasonic transducers are configured to deliver an ultrasound frequency of between 0.5 and 10 MHz, preferably between 1 and 4 MHz.

In one embodiment, the at least two ultrasonic transducers are configured to deliver ultrasound at a power of between 50 and 800 mW/cm².

Advantageously, the frequency and power of the ultrasound are chosen such that sufficient energy is transferred to the skull of the subject in order to cause the injected microbubbles to oscillate at the level of at least one targeted site of the brain thus allowing the blood-brain barrier to be selectively broken by mechanical action.

### DEFINITIONS

In the present invention, the terms below are defined as follows:
“Administration” or one of its variants (for example “administer”), means supplying the active agent or the active principle, alone or in a pharmaceutically acceptable composition to the patient in whom the condition, symptom or disease is to be treated or prevented. The expression “pharmaceutically acceptable” designates the ingredients of a pharmaceutical composition which are compatible with one another and which are not harmful for the subject to which they are administered.
“Anti-inflammatory” concerns a substance used to fight against inflammation, the body's defense process against aggression, characterized by signs of heat, pain, redness and swelling. In one embodiment, the anti-inflammatory is steroidal (cortisone type). In one embodiment, the anti-inflammatory is nonsteroidal.
“Anti-TNF alpha” relates to medicinal products resulting from biotherapy (also called biomedicines or biosimilars in relation to a reference anti-TNF alpha). In one embodiment, the anti-TNF alphas are fully or partially humanized monoclonal antibodies. In one embodiment, the anti-TNF alphas are chimeric proteins that behave as soluble TNF alpha receptors. Anti-TNF alphas reduce serum TNF alpha, which will make it possible to control regional inflammation and therefore the development of these pathologies. In one embodiment, they are administered repeatedly by intravenous infusions. In one embodiment, they are administered by subcutaneous injections in combination or as monotherapy. In one embodiment, the anti-TNF alphas are chosen from Etanercept, Infliximab, Adalimumab, Golimumab, Certolizumab.
“Bioavailability” refers to the proportion of a substance that reaches the bloodstream in unchanged form. Within the meaning of the invention, relates to the proportion of anti-inflammatory, in particular of anti-TNF alpha reaching the cerebral tissues in unchanged form.
The terms “therapeutically effective amount” or “effective amount” or “therapeutically effective dose” refer to the amount or dose of active ingredient that is intended, without causing significant negative or undesirable side effects to the subject, to (1) reduce the severity or incidence of disease; (2) slowing or stopping the progression, aggravation or deterioration of one or more symptoms of the targeted disease affecting the subject; (3) bring about improvements in the symptoms of the targeted disease affecting the subject; or (4) cure the targeted disease affecting the subject.
“About”, placed in front of a number, means plus or minus 10% of the nominal value of this number.
“Inflammation mediator” concerns chemical mediators that trigger and stimulate the inflammatory reaction. It is these mediators that are responsible for the characteristic manifestations of inflammation: increased vascular permeability, vasodilation, fever and pain. Non-limiting examples of inflammatory mediators are histamine, prostaglandins, pro-inflammatory cytokines (TNF, IL1, and IL6).
“Sagittal plane” concerns a plane going from front to back and forming a right angle with the frontal plane. It is parallel to the median plane.
“Frontal plane” concerns a plane perpendicular to the sagittal plane and which separates the body into an anterior or ventral part and a posterior or dorsal part.
“Median plane” concerns a plane which separates the left half and the right half of the body.
“Treat”, “cure” and “treatment”, as used in the present invention, refer to therapeutic treatment, excluding prophylactic or preventive measures, in which the objective is to slow down (mitigate) a given disease. People in need of treatment include those who already have the disease as well as those suspected of having the disease. A subject is successfully “treated” for a given disease if, after being treated with the method for improving the bioavailability of anti-inflammatory mediator agents in a subject or the method of treatment according to the present invention, said subject has an observable and/or measurable reduction or absence of one or more of the following: one or more of the symptoms associated with central nervous system disease; reduced morbidity and mortality; and/or improved quality of life. The above parameters for evaluating treatment success and improvement in central nervous system disease are readily measurable by routine procedures familiar to a physician.
“Subject” designates a mammal, preferably a human being. In one embodiment, a subject may be a “patient”, i.e., a mammal, more preferably a human, who is awaiting, or is receiving medical care, or has been/is/will be the undergoing a medical procedure, or is being monitored for the development of a disease.
The term “mammal” here means any mammal, including humans, domestic and farm animals, zoo animals, sporting animals, etc., such as dogs, cats, cattle, horses, sheep, pigs, goats, rabbits, etc. Preferably the mammal is a primate, more preferably a human. The term “human” means a subject of either sex and at any stage of development (ie newborn, infant, minor, adolescent, adult). In one embodiment, the subject is a male. In another embodiment, the subject is a woman. In one embodiment, the subject is an adult. In another embodiment, the subject is a child.
“Ultrasonic transducer” relates to a system converting electrical energy into acoustic energy in the ultrasonic range. In one embodiment, the ultrasound transducer is a micromachined transducer.
“Micromachined transducer” concerns a transducer manufactured using microsystems technologies. In one embodiment, the micromachined transducer has a dimension between 10 and 100 microns. In one embodiment, the ultrasound transducer is a capacitive micromachined transducer (CMUT). In one embodiment, the ultrasonic transducer is a piezoelectric micromachined transducer (PMUT).
“Ultrasound” concerns a mechanical and elastic wave, which propagates through fluid, solid, gaseous or liquid supports. In one embodiment, the ultrasound frequency range is between 16,000 and 10,000,000 Hertz.
“Diagnostic intensity” concerns an ultrasound intensity of less than 800 mW/cm², more precisely between 50 mW/cm² and 800 mW/cm².

### BRIEF DESCRIPTION OF THE FIGURES

FIG. 1 is a schematic representation of the ultrasound emission device according to an embodiment of the present invention.

FIG. 2 is a schematic representation of the ultrasound emission device according to another embodiment of the present invention.

FIG. 3 is a schematic representation of the ultrasound emission device according to another embodiment of the present invention.

FIG. 4 is a flow chart showing the main steps of the method according to the present invention.

### REFERENCES

1—ultrasound emitting device

2—structure

3—ultrasonic transducer

4—control device

### DETAILED DESCRIPTION

The following description will be better understood upon reading the drawings. In order to illustrate the invention, the device is shown in preferred embodiments. It should be understood, however, that the present application is not limited to the arrangements, structures, features, embodiments and precise appearance indicated. The drawings are not drawn on the scale and are not intended to limit the scope of the claims to the embodiments shown in these drawings. Therefore, it should be understood that when features mentioned in the claims are followed by references, said references are included only in order to improve the understanding of the claims and do not limit any case in the scope of these claims.

### Device

The present invention relates to an ultrasound emitting device 1, configured to selectively and reversibly break the blood-brain barrier of a subject, comprising a structure 2 configured to be placed on at least a portion of the head of said subject and at least two ultrasonic transducers 3 coupled to said structure 2, configured to emit focused ultrasound with diagnostic intensity. In one embodiment, said device 1 is adapted to increase the bioavailability in brain tissues of at least one agent against inflammation mediators. In a preferred embodiment, said device 1 is suitable for increasing the bioavailability in brain tissues of at least one anti-inflammatory agent. In a still more preferred embodiment, said device 1 is adapted to increase the bioavailability in brain tissues of at least one anti-TNF alpha.

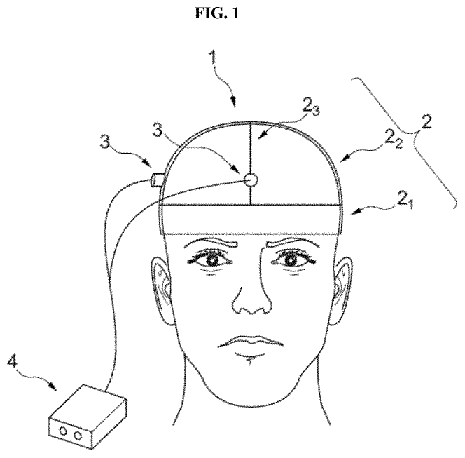

FIG. 1 shows an embodiment according to the invention in which the device 1 comprises a structure 2 consisting of a band adapted to surround the head of a subject, preferably the band consists of a circular band 2₁ configured to encircle the head of the subject at the temples, a first upper strip 2₂ connected to the circular band along a frontal plane and a second upper strip 2₃ connected to the circular band along a sagittal plane. The two upper bands 2₂ and 2₃ are configured to rest on the top of the head of the subject. In this embodiment, two ultrasonic transducers 3 are coupled to the band 2. A first temporal transducer is coupled to the first front upper band 2₂ and the second front transducer is coupled to the second upper sagittal band 2₃. In this embodiment, the two transducers 3 are movable and are positioned so as to scan the entire surface of the brain of the subject. The temporal transducer is movable in accordance with the choice of a frontal or transverse axis and the frontal transducer is movable along a sagittal axis. As shown in FIG. 1, the two transducers 3 rest on the surface of the scalp. As shown in FIG. 1, the ultrasonic transducers 3 are coupled to a control device 4 configured to control the frequency and power of the ultrasound emitted by said ultrasonic transducers 3. In this embodiment, the control device 4 is coupled to the ultrasonic transducers 3 by an electric cable.

According to one embodiment, at least two ultrasonic transducers are coupled to the structure 2. In one embodiment, at least one ultrasonic transducer is coupled to the structure 2 according to a frontal plane. In one embodiment, at least one ultrasonic transducer is coupled to the structure 2 in a sagittal plane. In one embodiment, the at least two ultrasonic transducers are coupled to the structure 2 according to a frontal plane. In one embodiment, the at least two ultrasonic transducers are coupled to the structure 2 in a sagittal plane.

According to one embodiment, the at least two ultrasonic transducers are movable. In one embodiment, the at least two ultrasonic transducers are movable along a frontal axis. In one embodiment, the at least two ultrasonic transducers are movable along a sagittal axis. In these embodiments, a scan on either side of the median plane is possible.

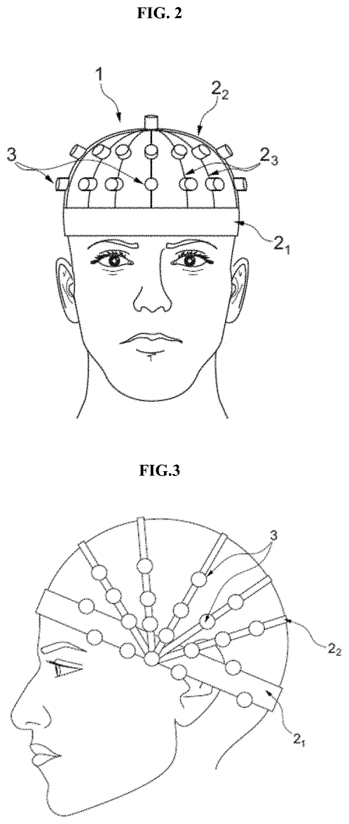

FIG. 2 shows another embodiment according to the invention in which the device 1 described in FIG. 1 is capable of receiving a plurality of ultrasonic transducers 3. According to one embodiment, a plurality of ultrasonic transducers 3 means from three to five ultrasonic transducers. According to one embodiment, a plurality of ultrasonic transducers 3 means at least three ultrasonic transducers. According to one embodiment, a plurality of ultrasonic transducers 3 means at least four ultrasonic transducers. According to one embodiment, a plurality of ultrasonic transducers 3 means at least five ultrasonic transducers. According to one embodiment, a plurality of ultrasonic transducers 3 means at least ten ultrasonic transducers. According to one embodiment, a plurality of ultrasonic transducers 3 means at least fifteen ultrasonic transducers. According to one embodiment, a plurality of ultrasonic transducers 3 means at least twenty ultrasonic transducers. According to one embodiment, a plurality of ultrasonic transducers 3 means at least fifty ultrasonic transducers. According to one embodiment, a plurality of ultrasonic transducers 3 means at least one hundred ultrasonic transducers. According to one embodiment, a plurality of ultrasonic transducers 3 means at least two hundred ultrasonic transducers. According to one embodiment, a plurality of ultrasonic transducers 3 means at least three hundred ultrasonic transducers. According to one embodiment, a plurality of ultrasonic transducers 3 means at least four hundred ultrasonic transducers. In this embodiment, the plurality of ultrasonic transducers 3 forms an array of transducers on the surface of the scalp. As shown in FIG. 2, the ultrasonic transducers are coupled to a plurality of strips in a sagittal plane.

In one embodiment shown in FIG. 3, the transducers of a plurality of ultrasonic transducers 3 are coupled to the structure 2 at the temporal zone. In this embodiment, the ultrasound emission is concentrated on the temporal zone of a subject.

According to one embodiment, the structure 2 is configured to entirely cover the head of a subject. In one embodiment, the structure 2 is configured to partially cover the head of a subject. In one embodiment, the structure 2 is configured to surround the head of a subject, preferably at the temples of said subject.

According to one embodiment, the structure 2 consists of a helmet. In one embodiment, the structure 2 consists of a cup. In one embodiment, the structure 2 consists of a hood. According to one embodiment, the structure 2 consists of a headband.

In one embodiment, the at least two ultrasonic transducers are selected from the capacitive micromachined ultrasonic transducers (CMUT), the piezoelectric micromachined ultrasonic transducers (PMUT) or the piezoelectric transducers. In a preferred embodiment, the at least two ultrasonic transducers are of the capacitive micromachined type (CMUT) or piezoelectric micromachined ultrasound (PMUT). In a preferred embodiment, the at least two ultrasonic transducers are of the capacitive micromachined type (CMUT).

According to one embodiment, the at least two ultrasonic transducers consist of at least one bar comprising at least two elements. In one embodiment, the at least two ultrasonic transducers are isolated elements.

According to one embodiment, the control device 4 is external to the device 1. In one embodiment, the control device 4 is coupled to the at least two transducers 3 by an electrical cable. In one embodiment, the control device 4 is integrated with the at least two transducers 3.

In one embodiment, the control device 4 is configured to generate an electrical signal making it possible to activate the at least two ultrasonic transducers 3.

In one embodiment, the control device 4 is configured to generate signals separately in order to independently activate each of said at least two ultrasonic transducers 3. In one embodiment, the control device 4 is configured to generate a single signal enabling simultaneous activation of all the ultrasonic transducers 3.

In one embodiment, the control device 4 is configured to control different parameters of the at least two ultrasonic transducers 3. In one embodiment, the control device 4 is configured to control the frequency of the ultrasound emitted by the at least two ultrasonic transducers 3. In one embodiment, the control device 4 is configured to control the power of the ultrasound emitted by the at least two ultrasound transducers 3. In one embodiment, the control device 4 is configured to control the amplitude of the ultrasound emitted by the at least two ultrasound transducers 3.

According to one embodiment, the at least two ultrasonic transducers are configured to deliver ultrasound at a low frequency. In one embodiment, the at least two ultrasonic transducers are configured to deliver an ultrasound frequency of between 0.5 and 10 MHz. In one embodiment, the at least two ultrasonic transducers are configured to deliver an ultrasound frequency of between 1 and 4 MHz. In one embodiment, the at least two ultrasonic transducers are configured to deliver an ultrasound frequency of between 0.5 and 1 MHz. In one embodiment, the at least two ultrasonic transducers are configured to deliver an ultrasound frequency of between 4 and 10 MHz. In a preferred embodiment, the at least two ultrasonic transducers are configured to deliver an ultrasound frequency of about 1.5 MHz.

According to one embodiment, the at least two ultrasonic transducers are configured to deliver ultrasound at a power of between 50 and 800 mW/cm². In one embodiment, the at least two ultrasonic transducers are configured to deliver ultrasound at a power of between 50 and 100 mW/cm². In one embodiment, the at least two ultrasonic transducers are configured to deliver ultrasound at a power of between 100 and 400 mW/cm². In one embodiment, the at least two ultrasonic transducers are configured to deliver ultrasound at a power of between 400 and 600 mW/cm². In one embodiment, the at least two ultrasonic transducers are configured to deliver ultrasound at a power of between 600 and 800 mW/cm². In a preferred embodiment, the at least two ultrasonic transducers are configured to deliver ultrasound at a power of about 800 mW/cm².

In one embodiment, the at least two ultrasonic transducers 3 rest on the surface of the scalp of a subject. In this embodiment, a uniform pressure is exerted on the head of the subject thus making it possible to minimize energy loss and the effect of heating due to the emitted ultrasound.

### Method

The invention also relates to a method for improving the bioavailability of agents against inflammation mediators in a subject. For this purpose, the method is configured to emit focused ultrasound onto at least one specific site of the brain of a subject to selectively and reversibly break the blood-brain barrier.

Thus, the invention relates to a method for improving bioavailability in at least one region of the brain tissue of at least one agent against inflammation mediators in a subject in need thereof comprising the steps of: administering to said subject a therapeutically effective amount of at least one agent against inflammation mediators, injecting microbubbles of gas to said subject, applying the device of the present invention to at least a portion of the head of said subject, and emitting focused ultrasound onto at least one site of the brain of said subject with said device, preferentially over the entire brain tissue.

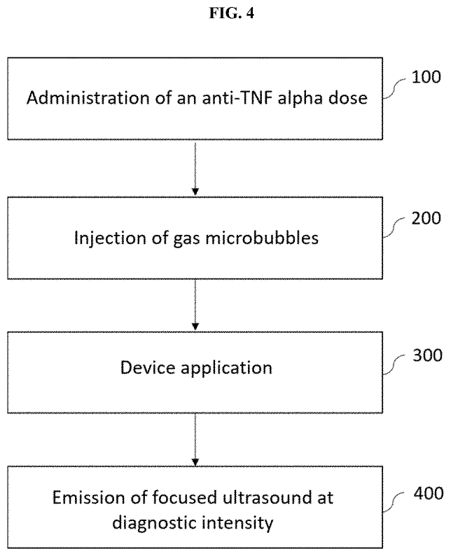

As shown in FIG. 4, the method of the present invention comprises four main steps. In a first step 100, a dose of agents against inflammation mediators is administered to a subject. The second step 200 consists in injecting microbubbles of gas to said subject. In a third step 300, the device of the present invention is applied to at least a portion of the head of said subject, preferentially to the surface of the skull of said subject. Finally, the fourth step 400 consists in emitting focused ultrasound onto at least one site of the brain.

According to one embodiment, the step of administering an anti-TNF alpha dose 100 is carried out before the injection of gas microbubbles 200, which is itself carried out before or concomitantly with the emission of focused ultrasound with diagnostic intensity 400 on at least one site of the brain of said subject. Step 300 during which the device of the present invention is applied to at least a portion of the head of said subject can be performed prior to administration of an anti-TNF alpha dose 100, between administration of an anti-TNF alpha dose 100 and the injection of gas microbubbles 200, or between injection of gas microbubbles 200 and the diagnostic intensity focused ultrasound emission 400 on at least one site of the brain of said subject.

In one embodiment, a dose of at least one agent against inflammation mediators is administered to the subject undergoing ultrasound. In one embodiment, a dose of at least one agent against inflammation mediators is administered to the subject at least once a week. In one embodiment, a dose of at least one agent against inflammation mediators is administered to the subject at least twice per week. In one embodiment, a dose of at least one agent against inflammation mediators is administered to the subject at least three days per week. In one embodiment, a dose of at least one agent against inflammation mediators is administered to the subject at least four days per week. In one embodiment, a dose of at least one agent against inflammation mediators is administered to the subject at least five days per week. In one embodiment, a dose of at least one agent against inflammation mediators is administered to the subject at least six days per week. In one embodiment, a dose of at least one agent for controlling inflammation mediators is administered to the subject seven days per week.

In one embodiment, a dose of at least one agent against inflammation mediators is administered to the subject for at least 24 weeks. In one embodiment, a dose of at least one agent against inflammation mediators is administered to the subject for at least 2 weeks. In one embodiment, a dose of at least one agent for controlling inflammation mediators is administered to the subject for at least 4 weeks. In one embodiment, a dose of at least one agent against inflammation mediators is administered to the subject for at least 8 weeks. In one embodiment, a dose of at least one agent for controlling inflammation mediators is administered to the subject for at least 12 weeks.

In one embodiment, a dose of at least one agent against inflammation mediators is administered to the subject at least once per week and for at least 24 weeks.

In practice, the therapeutically effective dose of agents against inflammation mediators to be administered depends on one or more parameters, including, in particular, the material used for administration, age, sex, size, weight, physical condition and degree of severity of the disorder to be treated. A person skilled in the art knows the therapeutically effective dose of anti-TNF alpha agents to be administered for controlling inflammation mediators.

In one embodiment, a dose of at least 25 mg of at least one agent against inflammation mediators is administered to the subject. In one embodiment, a dose of 25 mg of at least one agent against inflammation mediators is administered to the subject. In one embodiment, a dose of 50 mg of at least one agent for controlling inflammation mediators is administered to the subject. In one embodiment, the dose of at least one agent against inflammation mediators is administered by subcutaneous injection.

According to a preferred embodiment, said at least one agent for controlling inflammation mediators is an anti-inflammatory agent. According to a more preferred embodiment, said at least one agent for controlling inflammation mediators is an anti-TNF-alpha.

In one embodiment, the anti-TNF alpha is selected from Etanercept, Infliximab, Adalimumab, Golimumab, Certolizumab. In a preferred embodiment, the anti-TNF alpha administered is Etanercept.

According to one embodiment, the gas microbubbles are injected into said subject intravenously. In one embodiment, the microbubbles of gas injected into said subject are microbubbles of air, carbon dioxide, nitrogen, sulphur hexafluoride or perfluorocarbon such as perfluorohexane, perfluoropropane, perfluoropentane, it being understood that these microbubbles can be stabilized or encapsulated.

In this embodiment, an ultrasound contrast agent is preferably injected into said subject to generate the microbubbles of gas in the blood. In one embodiment, a solution of sulfur hexafluoride is injected into said subject. In one embodiment, a solution comprising iodipamide di-ester or diatrizoate is injected into said subject. In one embodiment, a solution comprising perfluorooctyl bromide or perfluorocarbon such as perfluorohexane, octafluoropropane, dodecabopentane, perfluoropentane is injected into said subject. In one embodiment, a solution comprising Echovist®, Perflubron™, IDE, Sonavist®, Levovist®, Albunex®, EchoGen®, Optison®, SonoVue®, Definity®, Imagent™, Imavist®, Sonazoid, Quantison™, Myomap™ ou SonoGen® is injected into said subject.

In one embodiment, the microbubbles have an average diameter of about 2.5 μm. In one embodiment, the microbubbles have a diameter of between 2 μm and 11 μm. In one embodiment, the microbubbles have a diameter of between 2 μm and 6 μm.

According to one embodiment, the device of the present invention is applied so as to entirely cover the head of a subject. In one embodiment, the device of the present invention is applied to partially cover the head of a subject. In one embodiment, the device of the present invention is applied so as to cover or surround the head of a subject, preferably at the temples of said subject. In one embodiment, the device of the present invention is applied so that the at least two ultrasonic transducers rest on the surface of the scalp.

In one embodiment, said subject is subjected to ultrasound focused on at least one specific site of the brain of said subject. In one embodiment, the subject is subjected to ultrasound for a period of between 5 min and 1 hour. In one embodiment, said subject is subjected to ultrasound for a period of between 5 min and 30 min. In one embodiment, said subject is subjected to ultrasound for a period of between 30 min and 1 hour. In one embodiment, said subject is subjected to ultrasound for a period of at least 30 minutes. In a preferred embodiment, said subject is subjected to ultrasound for a period of 30 minutes.

In one embodiment, said subject is subjected to ultrasound at least once per day. In one embodiment, said subject is subjected to ultrasound at least twice per day. In one embodiment, said subject is subjected to ultrasound twice daily.

In one embodiment, said subject is subjected to ultrasound at least one day per week. In one embodiment, said subject is subjected to ultrasound at least two days per week. In one embodiment, said subject is subjected to ultrasound at least three days per week. In one embodiment, said subject is subjected to ultrasound at least four days per week. In one embodiment, said subject is subjected to ultrasound at least five days per week. In one embodiment, said subject is subjected to ultrasound at least six days per week. In one embodiment, said subject is subjected to ultrasound seven days per week.

In one embodiment, the subject is subjected to ultrasound for at least 2 weeks. In one embodiment, the subject is subjected to ultrasound for at least 4 weeks. In one embodiment, the subject is subjected to ultrasound for at least 8 weeks. In one embodiment, the subject is subjected to ultrasound for at least 12 weeks. In one embodiment, the subject is subjected to ultrasound for at least 24 weeks. In one embodiment, the subject is subjected to ultrasound for 24 weeks.

According to a preferred embodiment, the subject is subjected to ultrasound for 30 minutes, twice daily, at least five days per week and for at least 24 weeks.

In one embodiment, the subject is subjected to ultrasound at a frequency of between 0.5 and 10 MHz. In one embodiment, the subject is subjected to ultrasound at a frequency of between 1 and 4 MHz. In one embodiment, the subject is subjected to ultrasound at a frequency of between 0.5 and 1 MHz. In one embodiment, the subject is subjected to ultrasound at a frequency of between 4 and 10 MHz. In one embodiment, the subject is subjected to ultrasound at a frequency of 1.5 MHz.

In one embodiment, the subject is subjected to ultrasound at a power of between 50 and 800 mW/cm². In one embodiment, the subject is subjected to ultrasound at a power of between 50 and 100 mW/cm². In one embodiment, the subject is subjected to ultrasound at a power of between 100 and 400 mW/cm². In one embodiment, the subject is subjected to ultrasound at a power of between 400 and 600 mW/cm². In one embodiment, the subject is subjected to ultrasound at a power of between 400 and 800 mW/cm². In one embodiment, the subject is subjected to ultrasound at a power of about 800 mW/cm².

Advantageously, the frequency and power of the ultrasound are chosen such that sufficient energy is transferred to the skull of the subject in order to cause the injected microbubbles to oscillate and increase the vascular porosity at the level of at least one targeted site of the brain, thus allowing the blood-brain barrier to be selectively broken by mechanical action.

According to one embodiment, steps 200 to 400 are repeated as many times as necessary.

As described above, the device of the present invention enables selective and reversible breaking of the blood-brain barrier by emitting focused ultrasound onto at least one specific site of the brain of said subject. The localized disruption of the blood-brain barrier allows the dose of the modulator of the inflammation, that is to say agent against the mediators of inflammation, administered circulating in the blood to more effectively penetrate through the open region of the blood-brain barrier, thereby delivering the effective dose necessary for the treatment of the targeted disease.

According to one embodiment, the method for improving the bioavailability of agents against inflammation mediators according to the invention is used for the treatment of diseases of the central nervous system.

The present invention also relates to a method of treating central nervous system diseases using the method of the present invention for improving the bioavailability of agents against inflammation mediators in a subject in need thereof.

The present invention also relates to a method for treating diseases of the central nervous system using the device of the present invention.

According to one embodiment, the device of the present invention is used for the treatment of diseases of the central nervous system.

In one embodiment, the central nervous system disease is selected from Alzheimer's disease, Parkinson's disease, epilepsy, cerebrovascular diseases, including stroke, migraine, multiple sclerosis, nervous system infections, brain tumors, traumatic nervous system disorders such as cranial trauma, depression and malnutrition-related neurological disorders.

Although various embodiments have been described and illustrated, the detailed description should not be considered to be limited thereto. Various modifications can be made to the embodiments by a person skilled in the art without departing from the true spirit and scope of the disclosure as defined by the claims.

1-8. (canceled)

9. A device configured to selectively and reversibly break the blood-brain barrier of a subject by emitting ultrasound, comprising:
a structure configured to be placed on at least a portion of the head of a user;
at least two ultrasonic transducers coupled to said structure and configured to emit ultrasound with diagnostic intensity;
wherein said at least two ultrasonic transducers are of capacitive micromachined, piezoelectric micromachined or piezoelectric type and configured to deliver an ultrasound frequency of between 0.5 and 10 MHz, preferably between 1 and 4 MHz, said at least two ultrasonic transducers being adapted to increase the bioavailability in brain tissues of at least one agent against inflammation mediators, preferably at least one anti-inflammatory, even more preferably at least one anti-TNF alpha.

10. The device according to claim 9, wherein said structure consists of a helmet, a cup, a hood or a headband.

11. The device according to claim 9, wherein said at least two ultrasonic transducers are positioned in a frontal plane and/or a sagittal plane.

12. The device according to claim 9, wherein said at least two ultrasonic transducers are movable along a frontal axis and/or a sagittal axis.

13. The device according to claim 9, wherein said device further comprises a control device coupled to the at least two ultrasonic transducers and configured to control the frequency and power of the ultrasound emitted by the at least two ultrasonic transducers.

14. The device according to claim 9, wherein said at least two ultrasonic transducers rest on the surface of the scalp.

15. The device according to claim 9, wherein said device comprises a plurality of ultrasonic transducers forming an array of transducers on the surface of the scalp.

16. The device according to claim 9, wherein said at least two ultrasonic transducers are configured to deliver ultrasound at a power of between 50 and 800 mW/cm².
